(19) United States
(12) Patent Application Publication
(10) Pub. No.: US 2023/0226294 A1
(43) Pub. Date: Jul. 20, 2023

(54) METHOD AND APPARATUS OF SMARTLY CONTROLLED ENDOTRACHEAL TUBE

(71) Applicants: Chunsheng Mao, Vancouver (CA); Yingwei Wang, Richmond (CA)

(72) Inventors: Chunsheng Mao, Vancouver (CA); Yingwei Wang, Richmond (CA)

(73) Assignee: LANCING TECHNOLOGY INC., Vancouver (CA)

(21) Appl. No.: 17/984,412

(22) Filed: Nov. 10, 2022

Related U.S. Application Data
(60) Provisional application No. 63/299,927, filed on Jan. 15, 2022.

(51) Int. Cl. A61M 16/04

(52) U.S. Cl. CPC A61M 16/04; A61M 16/044

(57) ABSTRACT

A method and apparatus for a smartly controlled dual-cuff endotracheal tube. A fully automatic or manual closed-negative-feedback loop control logic inflates/deflates a dual-cuff mechanism inside a patient's trachea. Separate inflating and deflating units with independent air control functions control the pressure based on signals from the cuff pressure control system. The dual-cuff mechanism comprises an inner and outer cuff connected to pilot balloons with individual pressure sensors to measure pressure and send data to the control system to calculate the delta. The inner cuff has an opening that serves as a pressure equilibrium indicator for the optimal cuff pressure being achieved. The apparatus can be manufactured to be a disposable product after a single use. The disclosure achieves: increased control and ease of use; increased accuracy, range, and control of pressurized airflow; an improved way of determining ideal cuff pressures in real-time; improved hygiene; and decreased costs.

### CROSS-REFERENCE TO RELATED APPLICATION

This application claims the benefit of U.S. Provisional Patent Application No. 63/299,927, filed on Jan. 15, 2022. The entire disclosure of the above application is incorporated herein by reference.

### FIELD

The present disclosure is in the field of medical equipment, airway management, ventilator systems, breathing tubes, endotracheal tubes, cuff pressure monitoring, and especially, a method and apparatus of a dual-cuff endotracheal tube with smartly monitored and controlled cuff pressure.

### BACKGROUND

A tracheal tube is a flexible catheter that provides mechanical ventilation by creating and maintaining an airway for patients. Such a device is connected to a ventilator that provides oxygen to the lungs. One particular type of tracheal tube is called an endotracheal tube, which provides an airway to a patient by inserting it into the patient's trachea via the mouth or nose. These tubes typically have a cuff at a distal end to keep the tube in place and act as a seal to prevent air and secretions from leaking through. A user inflates the cuff using an inflation device like an inflation bag, syringe, machine, or any other suitable inflationary device. Such a device brings pressurized air through an inflation tube to expand the cuff.

However, current means of cuff pressure control have the following problems: (1) cuff pressure is generally controlled manually in existing products. This means that the user has to know the correct cuff pressure and use a manual inflationary and deflationary device; (2) pressure control for many endotracheal tubes is not electrical and automatic, therefore it is slow to operate. Furthermore, the cuff pressure cannot be viewed and modified in real-time. This means that the user would need to constantly monitor the patient; (3) pressure is controlled in an open-looped or unidirectional way, which leads to a low control accuracy, for example, underinflation or overinflation of the cuff. The present disclosure provides a novel, fully automatic, closed-loop, electrical, smart control of cuff pressure, which also includes a solution to the next problem with existing cuff pressure control described as follows, of course, the control can also be manual.

The ideal pressure for cuff inflation is needed to ensure that the cuff can sufficiently seal the trachea without damaging it. Hereinafter, the term ‘ideal’ may be used interchangeably with ‘optimal’ when referring to cuff pressure. Therefore, a high level of accuracy is needed, which makes the ideal cuff pressure hard to determine without prior knowledge and deep experience. As a result, determining the right cuff pressure is typically left to a user with prior knowledge and experience, such as a trained medical professional like a doctor or nurse. Furthermore, changed situations in an intubated patient (e.g., cuff leakage, tube misalignment, etc.) may call for determination and modification of cuff pressure in real-time. As a result, the cuff pressure may fall outside the ideal level at any point during intubation, leading to air leakage or trachea damage. For example, in the past, cuffs were overinflated to mitigate such a situation, but doing that caused long-term damage to the patients' tracheas, such as overstretching or disruption to lymphatic flow.

Some existing devices implement cuff pressure control to alert the user if the cuff needs to be inflated or deflated. This required the user to set a pressure range, triggering the alert if the cuff pressure falls outside this range. But this type of device still requires moderate monitoring, and manual control of the cuff is required when the alarm triggers. Some other existing devices also automatically manage cuff pressure, using a unit that inflates or deflates a cuff automatically. But there is less control in the level or degree of inflation/deflation of the cuff. In another sense, there is a chance that cuff inflation or deflation may be less accurate. Furthermore, determining the pressure range in the above-mentioned devices may be tricky to do accurately when ideal cuff pressure varies between patients and even between intubation periods within the same patient.

Some existing endotracheal tubes may have more than one cuff. Two or more single cuffs may be placed separately along the tube's body, but those are mainly for reinforcement and stability inside a patient's trachea than for determining ideal cuff pressure. Other existing endotracheal tubes have one cuff inside another, but it is mainly used for reinforcement or protecting a sensor inside the cuffs. Furthermore, the latter type of cuff arrangement typically requires separate inflation tubes—one for each cuff.

Analog or digital sensors are implemented inside the cuff to measure a variety of factors in intubation, mainly air pressure inside the cuff. Other variables measured in the cuff may include air flow or temperature. Typically, only one sensor is installed within the cuff, and in some cases, the sensor is integrated with a cuff pressure management device. Pressure within the cuff may not be as accurate when there is more than one cuff for the endotracheal tube.

Inflation and deflation of a cuff are often controlled with an integrated device. Typical devices used for this include a syringe, a cuff inflator, an inflation bag, or a machine designed for this purpose. But there might be limitations in the control of cuff inflation/deflation, particularly with manual devices. In the case where inflation and deflation are controlled by a machine, it may not even be possible to have both functions on at the same time to achieve continuous air pressure control. Therefore, a means for independent control of inflation and deflation is needed and provided by the present disclosure.

The present disclosure provides a method and apparatus for a dual-cuff endotracheal tube. The following improvements can be expected: (1) increased control ability and ease of use with a smart-controlled endotracheal tube apparatus; (2) increased range and control of pressurized air flowing into the dual-cuff mechanism; (3) improved accuracy for automatically determining ideal cuff pressure in real-time; (4) improved hygiene; (5) decreased costs.

### SUMMARY

The present disclosure provides a method and apparatus for a smartly controlled cuff endotracheal tube that is disposable, uses a closed negative feedback loop for cuff pressure control, and achieves an ideal cuff pressure in real-time. The purposes are to have better control for adjusting and maintaining cuff pressure and to have better cuff pressure control and accurate determination of the optimal cuff pressure required to completely seal a patient's trachea. The present disclosure comprises: a fully automatic or manual closed-loop electrical smart pressure control and/or a dual-cuff mechanism that can also determine the optimal cuff pressure in real-time; separate inflation and deflation units with independent air control functions for increased control accuracy and resolution of inflation/deflation; an endotracheal tube with a dual-cuff mechanism at the tube's distal end containing one inner cuff and one outer cuff. Each cuff is connected to a pressure pipe that extends outside of the tube, leading to a pilot balloon assembly comprising of an inner pilot balloon and an outer pilot balloon. The inner pilot balloon is connected to the inner pressure pipe that sends compressed air from the inflation unit to the inner cuff. The outer balloon and outer pressure pipe wrap around their interior counterparts respectively. Each pilot balloon contains its own pressure sensor to measure air pressure in the associated cuff. The inner cuff has at least one hole or opening, allowing for the simultaneous expansion of both cuffs; as well as a smart cuff pressure control system that receives and analyzes data and sends instructions to the inflation and deflation units.

One aspect of the present disclosure is the endotracheal tube apparatus operates through a fully automatic closed-loop electrical smart pressure control mechanism. This smart pressure control mechanism uses a negative feedback loop to accurately inflate or deflate the cuff(s) to a target cuff pressure required to optimally seal a trachea. The smart pressure control mechanism uses pressure measurements from pressure sensors inside the pilot balloon assembly. The cuff pressure control system analyzes the data and sends instructions based on smart functions to control inflation or deflation of the dual-cuff mechanism accordingly. The control is also electrically operated for automation and real-time determination and modification of cuff pressure. Of course, the control can be manual too.

Another aspect of the present disclosure involves increased control accuracy and resolution of inflation and deflation thanks to inflation and deflation units being separate and independent. To independently control the volume and speed of inflation and deflation, both the inflation and deflation units have discrete power steps that control the speed and volume of pressurized air flow. The units operate separately to achieve a greater number of combination possibilities for inflating and deflating control levels; they do so to a point where they can achieve a continuous inflation or deflation function. Ultimately, a net inflation or deflation function for pressurized air speed and volume can be achieved.

The third aspect is the dual-cuff mechanism to achieve ideally determined cuff pressure. As mentioned before, the distal end of the endotracheal tube has a dual-cuff mechanism comprising two cuffs—an inner and outer cuff with the inner cuff inside the outer cuff. Each cuff connects to a pressure pipe that extends outside the endotracheal tube to connect to a pilot balloon assembly. Each of the two pilot balloons has an individual pressure sensor to measure the air pressure inside the associated cuff. The sensors then send the detected data to the cuff pressure control system for analysis and generation of instructions for controls. The inflation tube directly sends pressurized air from the inflation unit to the inner pilot balloon. The pressurized air then travels to the inner cuff through the inner pressure pipe; the inner pressure sensor reads cuff pressure. The outer pressure pipe is mainly used to allow the outer pressure sensor to measure air pressure inside the outer cuff. The inflated inner cuff gets higher inflating pressure due to directly receiving pressurized air from the inflation unit via the inner pressure pipe leading to eventual contact with the inner surface of the outer cuff. At that point, the hole(s) of the inner cuff get sealed and cuff pressure in both cuffs has reached equilibrium (P1=P2 or ΔP=0). The inner cuff then inflates more and pushes the outer cuff further to seal the trachea, stopping at the measured pressure level reaches a threshold preset by the user. An ideal cuff pressure is then held and maintained at that level.

The inner cuff itself embodies the fourth aspect of the present disclosure, comprising at least one hole or opening on the inner cuff. The inner pressure pipe feeds air into the inner cuff for its expansion. The inner cuff hole or opening eventually touches the outer cuff, indicating that cuff pressure has reached equilibrium (ΔP=0). The inner cuff opening also provides simultaneous inflation of the dual-cuff mechanism, an aspect that is a property of the aforementioned aspect. As the inner cuff inflates, air slowly travels through the inner cuff opening to inflate the outer cuff. When the inner cuff opening touches against the outer cuff, the opening can be sealed and the air stops entering into the outer cuff anymore. There may be also a feature in the outer cuff at the place where it encounters the opening of the inner cuff that is designed to help seal the opening. At that point, the inflation of the inner cuff influences further outer cuff expansion until a threshold is reached.

In one of the simplified embodiments of the present disclosure, only a single cuff, i.e., the inner cuff is used. There is no outer cuff, outer pressure pipe, outer pilot balloon, and outer pressure sensor. The target cuff pressure is pre-determined or determined by the doctor or user in real-time. The control system will manage the target cuff pressure is achieved inside the single cuff that will seal the airway properly.

The last aspect is that the whole apparatus of the present method is made of disposable materials, for example, the inflation unit can be a compressed air unit, which is basically a plastic bag or other containers that contains a certain amount of high-pressure air; the deflation unit is simply an opening hole.

The components of the apparatus are made of a cost-effective material that is typically designed for a single use before disposal. This allows for mass production at a low cost. The electrical components are can also be made cheaply, for example, the processor unit can be made as an ASIC chip with a button a battery. All components are sterilized and sealed to be used when needed. The entire apparatus is then disposed of after intubation ends.

By using the method and apparatus provided, the overall performance and experience of intubating patients are improved by achieving the following: (1) increased control and ease of use of intubating patients thanks to the fully automatic closed-loop electrical smart pressure control mechanism and the increased control accuracy and resolution from separate inflation and deflation units; (2) increased range and control of pressurized air flowing into the dual-cuff mechanism thanks to the increased control accuracy and resolution of inflation/deflation from separate and independent inflation and deflation units; (3) improved accuracy for automatically determining ideal cuff pressure thanks to the smart dual-cuff mechanism and its inner cuff opening; (4) improved hygiene thanks to the disposability; (5) can be easily and cheaply manufactured.

### BRIEF DESCRIPTION OF THE DRAWINGS

The accompanying drawings, which are included to provide a further understanding of the invention and are incorporated in and constitute a part of this application, illustrate embodiment(s) of the present disclosure and, together with the description, serve to explain the principle of the invention. For simplicity and clarity, the figures of the present disclosure illustrate a general manner of construction of various embodiments. Descriptions and details of well-known features and techniques may be omitted to avoid unnecessarily obscuring the discussion of the present disclosure's described embodiments. It should be understood that the elements of the figures are not necessarily drawn to scale. Some elements' dimensions may be exaggerated relative to other elements for enhancing the understanding of described embodiments. In the drawings:

FIG. 1 illustrates an overview of a preferred embodiment of the dual-cuff endotracheal tube apparatus in the present disclosure.

FIG. 2 illustrates a block diagram depicting the various elements of the cuff pressure control system according to the preferred embodiment of the present disclosure.

FIG. 3 illustrates a horizontal cross-section of the endotracheal tube apparatus inside a trachea in an uninflated state and an inflated state respectively.

FIG. 4 illustrates vertical cross-section views of the dual-cuff mechanism's operations.

FIG. 5 illustrates a plot of pressure levels within the cuffs over time during cuff inflation.

FIG. 6 illustrates a block diagram representation of a general fully automatic closed-loop smart-controlled dual-cuff endotracheal tube.

FIG. 7 illustrates a flowchart outlining the intubation process of a patient using the preferred embodiment of the present disclosure.

FIG. 8 illustrates a flowchart outlining cuff inflation with the inflation and deflation units.

FIG. 9 illustrates a flowchart outlining the mechanism for reaching an ideal cuff pressure.

### DETAILED DESCRIPTION

The present disclosure provides a method and apparatus for a smartly controlled cuff endotracheal tube that achieves smart pressure control with a negative feedback loop and an ideal cuff pressure, both in real-time. Various examples of the present invention are shown in the figures. However, the present invention is not limited to the illustrated embodiments. In the following description, specific details are mentioned to give a complete understanding of the present disclosure. However, it may likely be evident to a person of ordinary skill in the art; hence, the present disclosure may be applied without mentioning these specific details. The present disclosure is represented as few embodiments; however, the disclosure is not necessarily limited to the particular embodiments illustrated by the figures or description below.

The language employed herein only describes particular embodiments; however, it is not limited to the disclosure's specific embodiments. The terms “they”, “he/she”, or “he or she” are used interchangeably because “they”, “them”, or “their” are considered singular gender-neutral pronouns. The terms “comprise” and/or “comprising” in this specification are intended to specify the presence of stated features, steps, operations, elements, and/or components; however, they do not exclude the presence or addition of other features, steps, operations, elements, components, or groups.

Unless otherwise defined, all terminology used herein, including technical and scientific terms, have the same definition as what is commonly understood by a person of ordinary skill in the art, typically to whom this disclosure belongs. It will be further understood that terms, such as those defined in commonly used dictionaries, should be interpreted as having the same meaning as defined in the context of the relevant art and the present disclosure. Such terms should not be construed in an overly strict sense unless explicitly described herein. It should be understood that multiple techniques and steps are disclosed in the description, each with its own benefit. Each technique or step can also be utilized in conjunction with a single, multiple, or all of the other disclosed techniques or steps. For brevity, the description will avoid repeating each possible combination of the steps unnecessarily. Nonetheless, it should be understood that such combinations are within the scope of the disclosure. Reference will now be made in detail to some embodiments of the present invention, examples of which are illustrated in the accompanying figures.

The cuff endotracheal tube method and apparatus operate through a closed-loop electrical smart pressure control mechanism. The endotracheal tube has one or more cuffs. This smart pressure control mechanism is the first aspect of the present disclosure. It uses a negative feedback loop created as a result of the connections and communication between the components of the endotracheal tube. In the preferred embodiment of the present disclosure, the smart pressure control uses all electrical components and connections, so it can monitor and automatically change the air pressure of the cuff in real-time. Communication between these components is done with data and instructions based on the execution of smart functions. In other words, the smart pressure control mechanism can automatically and accurately inflate or deflate the cuff accordingly based on smart functions carried out in the negative feedback loop. However, inflation/deflation may be done manually in another preferred embodiment, so the smart pressure control mechanism can guild a user to manually adjust the cuff pressure in real-time according to the negative feedback loop control.

A negative feedback loop, or aka balancing feedback, occurs when some function of the output of a system, process, or mechanism is fed back in a manner that tends to reduce the change in the output, whether caused by changes in the input or by other means that stabilizes the output. Negative feedback tends to promote a settling to equilibrium and reduces the effects of perturbations. Negative feedback loops in which just the right amount of correction is applied with optimum timing can be very stable, accurate, and responsive. Negative feedback is widely used in mechanical and electronic engineering, and also in many other fields ranging from chemistry and economics to physical systems such as the climate. General negative feedback systems are studied in control systems engineering.

The smart pressure control mechanism in the present disclosure uses a negative feedback loop by monitoring the air pressure inside the endotracheal tube cuffs with pressure measurements obtained from digital pressure sensors inside pilot balloons. The air pressure measurements are then sent to a cuff pressure control system. Specifically, two pressure transducers inside a control processor in the control system—one corresponding to each pressure sensor—receive this data and relay it to a microprocessor in the processor for analysis. The microprocessor compares the measured cuff pressure and the target pressure. If the measured cuff pressure is lower than the target pressure, the control system will enable an inflating function to increase the cuff pressure; the increased cuff pressure is supposed to be closer to the target pressure. If the measured cuff pressure is higher than the target pressure, the control system will enable a deflating function to decrease the cuff pressure; the decreased cuff pressure is also supposed to be closer to the target pressure. The above process is then repeated in multiple iterations and the absolute difference between the cuff pressure and target pressure should be getting smaller and smaller until converging to an equilibrium state. The equilibrium state means any more iteration will not make the measured cuff pressure any closer to the target pressure. Once the equilibrium state or a predefined threshold is reached, the negative feedback loop control stops.

The method and apparatus also involve an independent inflation unit and deflation unit with independent air control functions. Herein lies the second aspect, the increased air pressure control accuracy and resolution of inflation and deflation due to the independent inflation and deflation units with independent air control functions. Pressurized air flows from an air supply associated with the inflation unit to the dual-cuff mechanism via an inflation tube. The deflation unit has a release valve that allows air to escape from the pressurized air flowing through the inflation tube to escape or a powered vacuum to suck away air to reduce the pressure. If both the inflation and deflation units are turned on, they have their own discrete power steps that dictate the speed and volume of pressurized air flowing through the inflation and deflation tube. Both units carry out their respective air control functions independently at the required power steps to influence the volume and speed of pressurized air flowing through the inflation tube to the pilot balloons and, eventually, to the dual-cuff mechanism. In the preferred embodiment, the inflation and deflation units carry out their respective air control functions automatically. Both units receive control signals from the cuff pressure control system to turn on or off at the required power steps, according to pressure measurements from the pressure sensors in the pilot balloon assembly. The control signals sent by the cuff pressure control system dictate what power step each unit should be. So, the power steps from the inflation and deflation units create a net inflation or deflation function for a specific pressurized air speed and volume to the dual-cuff mechanism. As the cuff pressure is monitored in real-time, control signals will constantly instruct the inflation and deflation units to operate at particular power steps throughout the entire intubation process. In another preferred embodiment, cuff inflation/deflation is done manually. In such cases, the user will then be instructed by the cuff pressure control system to inflate or deflate the cuffs at the specific power levels needed at a given time during intubation. In general, because the units operate independently from each other, there is a greater plurality of combination possibilities, increasing the control accuracy and resolution for inflation and deflation to a point where inflation/deflation can be adjusted at a wider or close to a continuous range.

The third aspect of the present disclosure involves the endotracheal tube's dual-cuff mechanism comprising an outer cuff and an inner cuff. The inner cuff is located inside the outer cuff and both cuffs have a torus (donut) shape sharing the endotracheal tube going through their centers. The inner cuff has at least one opening to the outer cuff. There are two openings on the two opposite sides of the horizontal centerline of the inner cuff in the preferred embodiment of the present disclosure. Each cuff is connected to a pressure pipe—an inner pressure pipe is connected to the inner cuff and an outer pressure pipe is connected to the outer cuff and covers the inner pressure pipe. The pressure pipes extend outside the endotracheal tube to connect to a pilot balloon assembly—an outer pilot balloon connects to the outer pressure pipe and an inner pilot balloon connects to the pressure pipe. As noted earlier when describing the first aspect earlier, the air pressure inside each cuff is measured via pressure sensors inside the respective pilot balloons: a first sensor that is installed in the inner pilot balloon measures the inner cuff air pressure (P1); a second sensor that is installed in the outer pilot balloon measures the outer cuff air pressure (P2). The pressure measurement data is sent to the cuff pressure control system, where ΔP=(P1−P2) is calculated by the processor. Instructions for controls are then generated by the processor of the cuff pressure control system and are then sent to the inflation and deflation units automatically in the preferred embodiment. In an alternative embodiment, the information is displayed to the user to adjust the inflation and deflation units at the required power levels when both the inflation and deflation units are powered machines. During inflation, the inner cuff inflates first via the inner pressure pipe. The inner pilot balloon also inflates from the pressurized air while measuring air pressure inside the inner cuff. Meanwhile, the outer pressure pipe merely connects the outer pilot balloon to the outer cuff as a means to measure pressurized air inside the outer cuff. The inner cuff commonly gets a higher inflating pressure than the outer cuff (P1>P2) at this moment. After that, ΔP gradually decreases towards zero. During this time, the inner cuff expands to the point where it eventually touches the interior surface of the outer cuff and the outer cuff will be forced to expand too. When the outer cuff is expanding along with the inner cuff, the pressure from the outer cuff applied to the openings/holes increases. At one point in time, said pressure is high enough to block and seal the openings/holes. This indicates that there is no more air going from the inner cuff to the outer cuff. The pressure in the inner cuff (P1) is roughly equal to that of the outer cuff (P2). The equal pressure between the cuffs is considered the pressure equilibrium (P1=P2); the difference in pressure (ΔP) is zero. The dual-cuff mechanism stays in equilibrium for a brief moment if cuff inflation continues. As the inner cuff continues to expand a little further. Because the openings/holes are sealed now, ΔP starts rising again from zero, where P1 is once again greater than P2. ΔP eventual rises to meet at a predefined threshold ΔP′ in pressure. At this threshold, the dual-cuff mechanism stops inflating and is maintained at this particular cuff pressure P1′ and P2′ (ΔP′). It should be noted that the dual-cuff mechanism may stop inflating above the threshold level ΔP′; however, this is acceptable as long as ΔP is not significantly higher than the threshold ΔP′. In a sense, there is a threshold range between the minimum threshold value and what is considered beyond acceptable. If ΔP rises far above the acceptable threshold range, then the dual-cuff mechanism is deflated via the deflation unit until ΔP is within the acceptable threshold range. Once ΔP is at this threshold range, the cuff pressure is maintained and considered an ideal pressure level. In one embodiment of the present disclosure, this ΔP′ is the air pressure measured in the previous description of the negative feedback loop control.

In an alternative embodiment of the present disclosure, only one cuff, the inner cuff is used. Then all the outer cuff, outer pressure pipe, outer pilot balloon, and its pressure sensor do not exist. The target cuff pressure is pre-determined or determined by the doctor or user in real-time. The control system will manage the target cuff pressure is achieved inside the single cuff that will seal the airway properly.

There are two additional novelties that are properties dependent on the third aspect. The fourth aspect is at least one hole or opening on the inner cuff mentioned above. Hereinafter, this hole/opening is referred to as the ‘inner cuff opening’, which can be generally used to entail either one single opening or multiple openings on the dual-cuff mechanism. This opening serves two purposes: (1) its closure serves as an indication of approaching pressure equilibrium, as it is the first part that makes contact with the interior surface of the outer cuff; (2) it allows pressurized air to leak out of the inner cuff. As the inner cuff is expanding, the outer cuff also expands, albeit at a slower rate. When the inner cuff opening presses against the outer cuff, the opening can be sealed and the air stops entering into the outer cuff anymore. At this point, the outer cuff's expansion is driven by physical contact with the inner cuff. Herein lies the fifth aspect that is a property of both the third and fourth aspects, which is simultaneous inflation of the two cuffs within the dual-cuff mechanism. With this aspect, one inflation tube can sufficiently fill both cuffs. This is helpful when dealing with patients with different trachea sizes. As this adjustment is done as needed throughout the intubation process, it also helps in changed situations during the intubation of a single patient. As the pressure level is monitored in real-time, a user may also monitor the situation to determine whether the ideal pressure is reached.

Another aspect is that the entire apparatus in the preferred embodiment is designed to be disposable after a single use. For example, the endotracheal tube, cuffs, pressure balloons, and other applicable materials are made of plastic, which can be cheaply manufactured via injection molding. In one embodiment, the inflation unit is primarily a readily available inflation device that is also injection molded like an inflation air container bag, a syringe, or even an air inlet. The cuff pressure control system in one embodiment can be cheaply made as a battery-powered machine with a processor and various other components for analyzing data regarding cuff pressure. When all components are manufactured and assembled, the apparatus is sterilized and sealed to be used when needed. The entire apparatus is then disposed of after intubation ends. In addition to lower costs, this is considered more hygienic because they are sealed before use and there is no need to worry about sterilization of the apparatus after intubation.

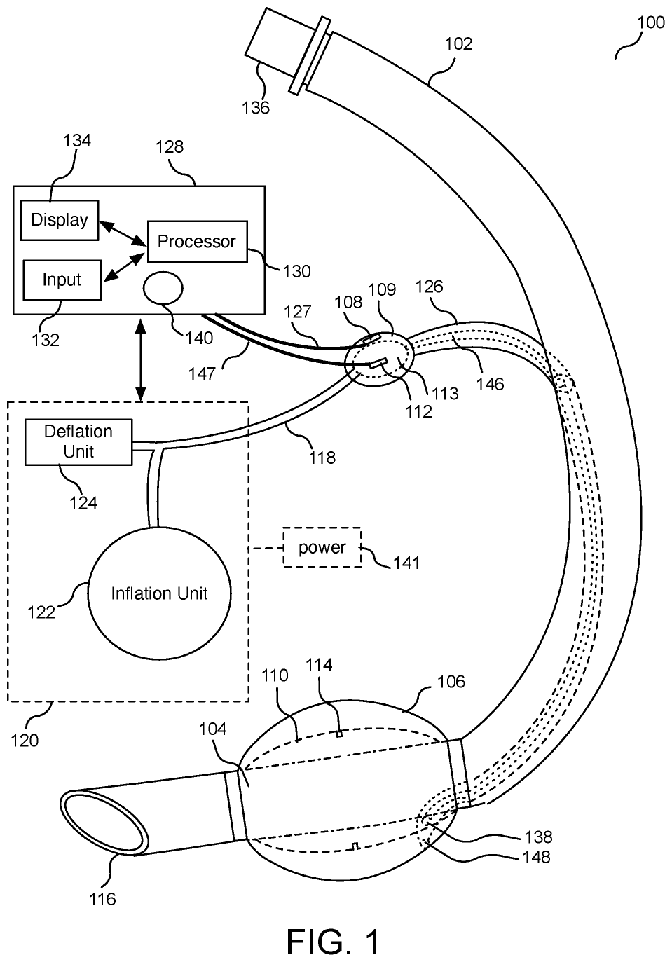

FIG. 1 illustrates a preferred embodiment of the dual-cuff endotracheal tube apparatus in the present disclosure. The endotracheal tube apparatus (100) comprises an endotracheal tube (102) with a long tubular body. Hereinafter, the term ‘endotracheal tube (102)’ may be used interchangeably with ‘tubular body (102)’. The proximal end of the endotracheal tube (102), shown at the top of the figure, has a connector (136), which connects the tubular body (102) to a ventilator to provide oxygen to the intubated patient. A dual-cuff mechanism (104) is located near the bevel (116) at the distal end of the endotracheal tube (102). The bevel (116) is an angled hole at the distal end of the tubular body (102). Oxygen exits from the tube to the patient via the bevel (116). Hereinafter, the term ‘bevel (116)’ is interchangeable with ‘distal end (116)’. The dual-cuff mechanism (104) comprises: an outer cuff (106) and an inner cuff (110). Each cuff (106, 110) is connected to an associated pressure pipe that travels along the body of the endotracheal tube (102) to extend outwards to connect to a pilot balloon assembly comprising an inner pilot balloon (113) and an outer pilot balloon (109): an inner pressure pipe (146) connects the inner pilot balloon (113) to the inner cuff (110); an outer pressure pipe (126) connects the outer pilot balloon (109) to the outer cuff (106). Each pilot balloon (109, 113) has a sensor that measures the air pressure within their respective cuff (106, 110): an outer balloon sensor or a first sensor (108) is attached to the interior of the outer pilot balloon (109); an inner balloon sensor or a second sensor (112) is attached to the interior of the inner pilot balloon (113). The inner cuff (110) has at least one hole or opening (114). Looking at the figure, one inner cuff opening (114) is on the top side and another inner cuff opening (114) is located at the bottom side of the inner cuff (110) when positioned along a transverse plane.

Pressurized air travels through an inflation tube (118) into the pilot balloon (109, 113) assembly. The inner pilot balloon (113) inflates as a result. The main inflation tube then transitions at the distal end of the pilot balloon (109, 113) assembly into an inner pressure pipe (146) with an outer pressure pipe (126) covering the inner pressure pipe (146). The pressurized air travels through the inner pressure pipe (146) to exit out the inner pressure pipe opening (138), filling the inner cuff (110) with air. The inner cuff opening (114) allows air to slowly leak out to inflate the outer cuff (106). The outer pressure pipe (126) branches away from the inner pressure pipe (146) near the dual-cuff mechanism (104). The outer pressure pipe (126) also has an outer pressure pipe opening (148), which mainly serves as a means to measure pressurized air inside the outer cuff (106).

The proximal end of the inflation tube (118) is connected to an inflation unit (122) that acts as the source of the pressurized air entering the dual-cuff mechanism (104). A deflation unit (124) is perpendicularly attached to the inflation tube (118). Both the inflation unit (122) and deflation unit (124) execute their air control functions (120) in tandem based on control signals from a cuff pressure control system (128). In some embodiments, both the inflation unit (122) and deflation unit (124) operates via a power source (141) so that they can operate automatically. Yet in some other embodiments, the inflation unit (122) is simply a compressed air container that contains a certain amount of high-pressure air; and the deflation unit (124) is an opening or hole to let the air out.

The sensors (108, 112) are connected to the cuff pressure control system (128) via a first connecting wire (127) for the first sensor (108) and a second connecting wire (147) for the second sensor (112). The cuff pressure control system (128) comprises a processor (130) that receives pressure measurement data from the sensors (108, 112) and sends control signals to the inflation (122) and deflation (124) units. A user can interact with the cuff pressure control system (128) with input (132) and a display (134), both of which are linked to the processor (130). The cuff pressure control system (128) may be powered via a battery (140).

The endotracheal tube (102) is presumed to be of standard size, typically with a depth of 21-23 centimeters and an inner diameter of 6-8.5 millimeters. However, the listed sizes are for exemplary purposes and are not limited to that size. For example, smaller endotracheal tubes (102) may be needed in alternative embodiments for intubating children.

All components of the apparatus (100) communicate with each other to inflate or deflate the dual-cuff mechanism (104) accordingly using the smart pressure control mechanism or, more specifically, the smart functions associated with the smart pressure control mechanism. The first (108) and second (112) pressure sensors send information about the pressure inside their respective cuffs (106, 110) to the cuff pressure control system (128). More specifically, the inner and outer pressure pipes (126, 146) connect the pilot balloons (109, 113) and the cuffs (106, 110) together so that the sensors (108, 112) can get cuff pressure measurements. The data is then analyzed by the processor (130), which then sends control signals to the inflation (122) and deflation (124) units to execute their respective air control functions (120). In a sense, the components of this apparatus (100) communicate with each other to carry out the inflation/deflation of the dual-cuff mechanism (104) through the smart pressure control mechanism. Furthermore, the smart pressure control mechanism is electrical in the preferred embodiment, which means that the inflation/deflation of the dual-cuff mechanism (104) is carried out quickly and automatically. However, it is obvious to those ordinarily skilled in the art this inflation/deflation control can be done with manual actions as well via the input (132) from the cuff pressure control system (128) and through manual operation of both the inflation (122) and deflation (124) units (in another embodiment). This smart pressure control mechanism aspect will be elaborated further in future figures.

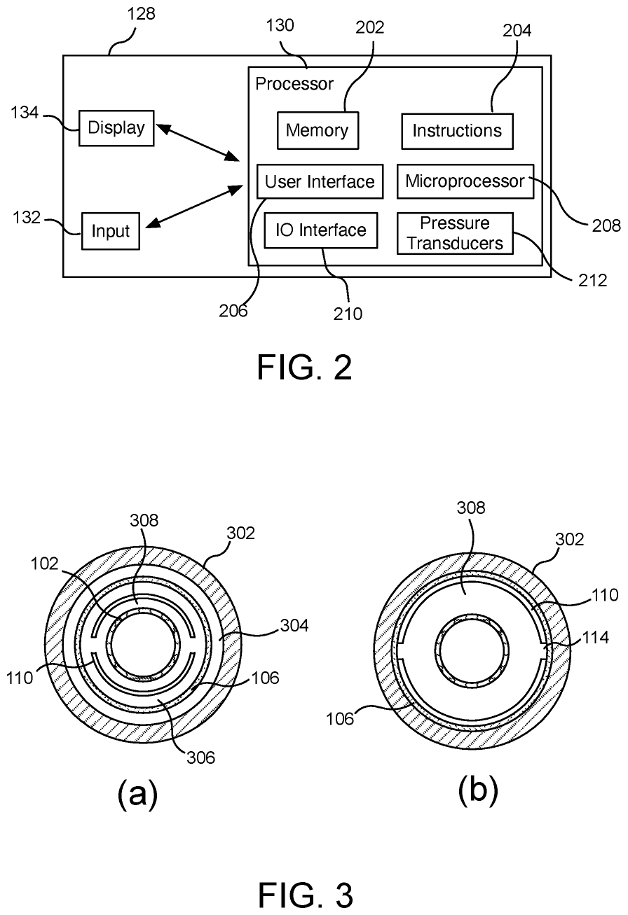

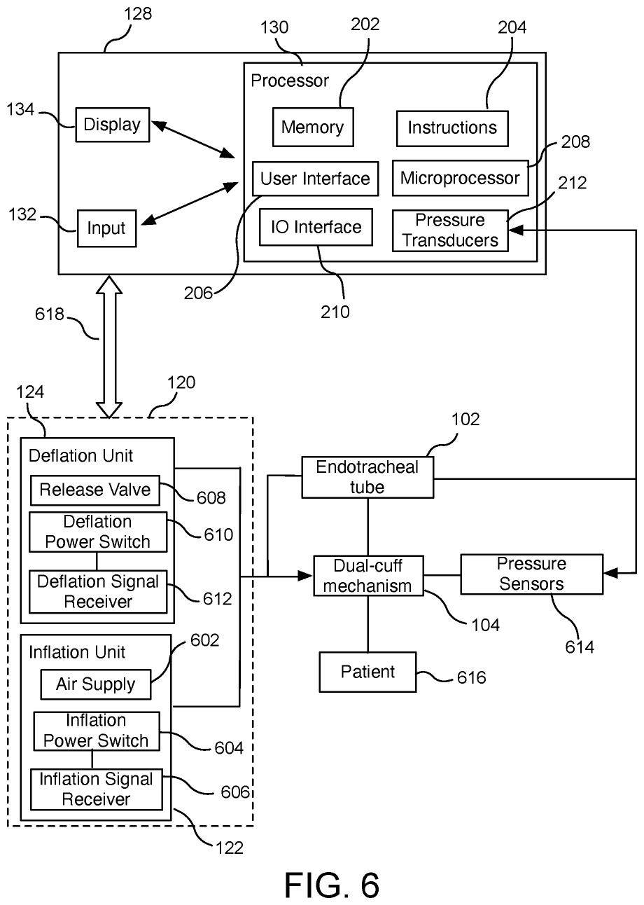

The inflation unit (122), deflation unit (124), and the cuff pressure control system (128) are generalized representations in this figure. The components all have additional elements that allow them to carry out the aspects involving the smart pressure control mechanism and the increased control accuracy and resolution. Such elements will be shown and explained further in future figures FIGS. 2 and 6.

The presence of two pressure sensors (108, 112) to measure cuff pressure in each individual cuff (106, 110) gives a more accurate reading of cuff pressure within the dual-cuff mechanism (104) as a whole. The calculations from the pressure sensors' (108, 112) readings are then used to inflate or deflate the dual-cuff mechanism (104) accordingly. The dual-cuff mechanism (104) operates alongside the smart pressure control mechanism aspect and the aspect involving the increased control accuracy and resolution of inflation/deflation to automatically determine and finely adjust the amount of pressurized air needed to inflate the dual-cuff mechanism (104) accordingly. As a result, the dual-cuff mechanism (104) can seal the trachea to the ideal cuff pressure more accurately without damaging the trachea. This will be shown and explained further in future figures.

The pressure sensors (108, 112) in the preferred embodiment are specifically designed to measure pressure. However, pressure is used as an exemplary unit of measure, and the apparatus (100) is not limited to just measuring pressure in other embodiments. In an alternative embodiment, the sensors (108, 112) can measure other variables like air volume, flow, body temperature, etc. In another embodiment, additional sensors (108, 112) may be implanted within the pilot balloons (109, 113) to solely measure different variables, such as the ones mentioned prior. If other measurements were collected, they could be collected and analyzed by the cuff pressure control system (128), so that cuff inflation/deflation can be even more accurate with the smart pressure control mechanism. Additional functions for the cuff pressure control system (128) and the other components of the apparatus (100) may be required in such cases.

It should be noted that the number of pressure sensors (108, 112) shown in the figure is exemplary and is not limited to just two pressure sensors (108, 112). In other alternative embodiments, there can be more than two sensors (108, 112) in the pilot balloon (109, 113) assembly with each balloon (109, 113) having different numbers of sensors (108, 112).

The pressure sensors (108, 112) are placed along the inner surfaces of their respective pilot balloon (109, 113). The sensors (108, 112) are typically in-line with the interior surfaces of their respective pilot balloons (109, 113). It is obvious to those ordinarily skilled in the art that such sensors (108, 112) would have a cover like a piece of foil, film, or mount to protect the sensors (108, 112) and keep them in place. The inner pilot balloon (113) still inflates as pressurized air travels to the dual-cuff mechanism (104). Such a cover may help if the inner pilot balloon (113) makes contact with the outer pilot balloon (109).

It should be noted that the placement of the sensors (108, 112) in the figure is for exemplary purposes and is not limited to that area in the pilot balloons (109, 113). In another embodiment, the sensors (108, 112) may be placed anywhere inside the respective cuffs (106, 110). This, however, may require the connecting wires (127, 147) to travel along the endotracheal tube's (102) body. Furthermore, there is also a risk of the sensors being accidentally placed inside the intubated patient if the apparatus (100) was defective and broke. In another alternative embodiment, the pressure sensors (108, 112) are placed on the outer surfaces of their respective cuffs. However, there is an even greater risk of the sensors (108, 112) being displaced during intubation. In yet another alternative embodiment, the pressure sensors (108, 112) would be integrated with the cuff pressure control system (128), interacting particularly with the processor (130).

The sensors (108, 112) in the present disclosure take the form of a thin, flat, chip that is presumably made of a flexible material like FPC. This type of material is flexible and would be able to fit along the interior surfaces of the inner (110) and outer (106) cuffs to let the dual-cuff mechanism (104) operate. Furthermore, FPC is a type of material that can be made cheaply in large quantities, making it cost-effective and suitable for disposability after a single use. However, it should be noted that this type of material is exemplary, so the sensors (108, 112) are not limited to only this type of material. In an alternative embodiment, the pressure sensors (108, 112) may take the form of probes protruding into each cuff (106, 110). Such probes may provide more accurate readings compared to a small microchip, but they are less compact. The probes would also be reusable, but would more expensive and not suitable for the disposable nature of the apparatus. Furthermore, protruding probes in the cuffs (106, 110) may affect how the dual-cuff mechanism (104) operates, mainly that the inner cuff (110) would not be able to come into proper contact with the outer cuff (106). As such, pressure equilibrium and ideal cuff pressure may not be achievable due to the large amount of space being taken up by the probes.

It should be noted that the pressure sensors (108, 112) take a rectangular form in the figure, but this is mainly for exemplary purposes. The pressure sensors can a circular, triangular, or any other suitable shape depending on the embodiment. The size of the pressure sensors (102, 112) is also exemplary and can vary in size depending on the embodiment.

The preferred embodiment of the present disclosure is shown with a dual-cuff mechanism (104) with two cuffs—one inner cuff (110) and one outer cuff (106). However, it should be noted that this is primarily exemplary, so the number of cuffs (106, 110) for the mechanism (104) may vary. In one embodiment, only a single cuff (106, 110) is used. In other embodiments, three or more may be used.

The dual-cuff mechanism (104) is shown at the distal end (116) of the endotracheal tube (102) in the preferred embodiment of the present disclosure. While it is standard for endotracheal tubes (102), this can be exemplary and is not limited to being placed at the distal end (116). The dual-cuff mechanism (104) may be placed anywhere along the endotracheal tube (102) in other alternative embodiments.

The inner cuff opening (114) serves as an indicator that pressure equilibrium between the cuffs (106, 110) is approaching. In a sense, the inner cuff opening (114) helps the dual-cuff mechanism (104) achieve the ideal cuff pressure. Outside the pressure equilibrium (P1=P2), the inner cuff's (110) pressure (P1) will always be higher pressure than the outer cuff's (106) pressure (P2). So, outside the pressure equilibrium, P1>P2. The inner pressure pipe opening (138) feeds pressurized air directly into the inner cuff (110). On the other hand, the inflation of the outer cuff (106) is dependent on the inner cuff's (110) inflation, whether it's by air leaking through the inner cuff opening (114) or the inner cuff (110) pushing on the interior surface of the outer cuff (106) after reaching pressure equilibrium. This also contributes to achieving ideal cuff pressure, as the outer cuff (106) does not expand so quickly inside the trachea. In a sense, the inner cuff opening (114) finely adjusts the rate of outer cuff (106) expansion. This will be shown further in future figures.

Because of the inner cuff opening (114), both cuffs (106, 110) can inflate/deflate simultaneously, as only the inner pressure pipe (146) is required to inflate both cuffs (106, 110). There are two inner cuff openings (114) shown in this figure on opposite sides of the inner cuff (110). However, this is shown for exemplary purposes, so the number of cuff openings (114) and/or cuff opening locations is not limited to the 470 amount shown in the figure. In other alternative embodiments, there may be only one or multiple small openings or one continuous opening at the center of the inner cuff (110) along a transverse plane (i.e., the horizontal center). As pressurized air escapes from the inner pressure pipe opening (138), the air travels in all directions to equally inflate the inner cuff (110) and by extension, the outer cuff (106).

The cuff pressure control system (128) in the preferred embodiment appears to take a form of a computer-like device. As the cuff pressure control system (128) is electrically controlled via a battery (140), such a device would be most beneficial in monitoring cuff pressure in real-time and automatically adjusting cuff pressure in the dual-cuff mechanism (104) at any time throughout the intubation process. However, the above description for the cuff pressure control system (128) is exemplary and is not limited to just this type of device. In another embodiment, where manual control is required, the cuff pressure control system (128) would display information to a user, so that they can adjust the inflation (122) and deflation (124) units' power levels manually.

The battery (140) in the preferred embodiment is typically a disposable type such as a standard single-cell battery (AA or AAA battery) or one of the button batteries. The battery would already be packaged with the rest of the apparatus (100) and be thrown out after a single use. In other embodiments, other sources of power in other embodiments may include a rechargeable lithium-ion battery, AC or DC electricity, USB connection, solar power, etc. However, such forms of power supply may be more expensive and affect the disposability aspect of the entire apparatus (100).

The power source (141) controlling the inflation unit (122) and deflation unit (124) may be the same power source of (140) or another simple battery, a rechargeable battery, AC or DV electricity, USB, or any other applicable source depending on the embodiment. In some embodiments, like a disposable apparatus, no power source (141) is present, and the inflation (122) and deflation (124) units operate manually. In such a case, the cuff pressure control system (128) merely displays data on the display (134) for the user to manually adjust cuff pressure. No control signals would be sent to the inflation (122) and deflation (124) units.

The inflation unit (122) is shown as an exemplary representation and is not limited to one particular device. In one embodiment, manual devices, such as a syringe, an inflation bag, a cuff inflator, or even a simple air inlet hole can be used to inflate the dual-cuff mechanism (104). Such devices are ideal for the disposable nature of the entire apparatus (100) since they are sterile and sealed before use. Such devices are also made of plastic and injection molding. In another embodiment, the inflation unit (122) can be a more complex motor-driven machine like an electrical pump. It should also be noted that the number of inflation units (122) is only exemplified as one unit but can be more than one device in other alternative embodiments.

The deflation unit (124) is also shown as an exemplary representation and is not limited to one particular device. In one embodiment, the deflation unit (124) may include a simple outlet hole on the inflation tube (118), a solenoid valve with multiple ports, or any other suitable device. Such a device can be made cheaply and disposed of after a single use. In another embodiment, the deflation unit (124) can be a more complex machine like an electrical vacuum. It should also be noted that the number of deflation units (124) is only exemplified as one unit but can be more than one device in other alternative embodiments.

In yet another alternative embodiment, the air control functions (120) and associated inflation (122) and deflation (124) units would presumably be integrated with the cuff pressure control system (128). Such examples may include a syringe or cuff inflator with a pressure gauge. While it does allow for a user with prior knowledge to adjust the cuff pressure quickly, it would require constant monitoring and the cuff pressure generated may be less accurate. In such an embodiment, the power source (141) and battery (140) can be the same, which would mean the inflation unit (122), deflation unit (124), and cuff pressure control system (128) would share power with each other.

The endotracheal tube (102) is typically made with a plastic material such as polyvinyl chloride. This type of material can be manufactured cheaply via injection molding and can be disposed of after a single use. As a result, costs can be saved when manufacturing the endotracheal tube (102). Furthermore, because of its disposability, there is increased hygiene due to the fact that the endotracheal tube (102) is only used for a single patient. However, it should be noted that this type of material is exemplary and the tubular body (102) is not limited to this particular material in other embodiments. The tubular body (102) may be made of other types of materials, such as another type of polymer, rubber, steel, etc. Such materials may be more suitable for specialized uses. In another alternative embodiment, the endotracheal tube (102) may be wire-reinforced when intubation lasts for a long period of time (e.g., at least several hours).

The inner pressure pipe (146) is shown to be enveloped within the outer pressure pipe (126) in this embodiment, with the distal ends branching out as separate pressure pipes (126, 146) around the dual-cuff mechanism (104) area. In another embodiment, both pressure pipes (126, 146) can be purely separate entities along the tubular body (102) of the endotracheal tube.

Those ordinarily skilled in the art would find it obvious that a seal is present within the endotracheal tube (102) and dual-cuff mechanism (104) where the pressure pipes (126, 146) intersect. Such a seal would be required to ensure that there is no air leakage and that accurate cuff pressure readings can be obtained during the inflation/deflation of the dual-cuff mechanism (104).

The connector (136) at the proximal end of the endotracheal tube (102) is attached to a ventilator (not shown) that provides oxygen to an intubated patient. In a typical embodiment, the ventilator is a standalone device separate from the cuff pressure control system (128). In other alternative embodiments, a device with integrated ventilation and cuff pressure control functions may be used. The inflation (122) and deflation (124) units would be also integrated into such a device. The inflation tube (118) would be also connected to such a device rather than the inflation (122) and deflation (124) units as shown in this figure.

FIG. 2 illustrates a block diagram depicting the various elements of the cuff pressure control system according to a preferred embodiment of the present disclosure. As noted in the description of FIG. 1, the cuff pressure control system (128) primarily comprises a processor (130) that receives pressure measurement data from the sensors and sends control signals to the inflation and deflation units. The display (134) communicates with the processor (130) to display information to the user regarding cuff pressure, inflation/deflation time, or any other relevant information during intubation. The display (134) may take the form of an LCD monitor, touch screen, or any other appropriate visual output device depending on the embodiment. The input (132) communicates with the processor (130) to allow users to input commands to the processor (130) and cuff pressure control system (128) as a whole. The input (134) may consist of a mouse, keyboard, keypad, touch panel, or any other suitable input device depending on the embodiment.

The processor (130) comprises multiple elements that work together to carry out the functions of the cuff pressure control system (128) and, in particular, perform the smart control function for cuff pressure management of the dual-cuff mechanism. In a sense, the processor (130) is considered the ‘brain’ of the cuff pressure control system (128). The user interface (206) allows for user interactions with the cuff pressure control system (128) via input and output commands. The user interface (206) may take a user's input (132) and translate it to code or executable commands for the processor (130). The memory (202) stores information relevant to cuff pressure measurement. The memory (202) may take the form of RAM memory, flash memory, hard disk drive, or a solid-state drive depending on the embodiments. The processor (130) also has instructions (204) that execute commands related to the smart pressure control mechanism during inflation/deflation. The instructions (204) are carried out to the inflation and deflation units in the form of control signals. The instructions (204) also interact with other components in the cuff pressure control system (128). For example, instructions (204) can be sent to the display (134) to show relevant information to the user in real-time. Pressure transducers (212) receive pressure measurements from the sensors and convert the data into a signal (typically an electrical signal) for the microprocessor (206) to receive. The microprocessor (208) analyzes the pressure measurement data, which then determines the rate of cuff inflation/deflation (i.e., the power steps of inflation and deflation units). In a sense, the microprocessor comprises the arithmetic, logic, and control circuitry that lets the processor (130) carry out its functions. The microprocessor (208) may consist of, but is not limited to, hardware, software, circuit and circuit components, internal logic, any combination thereof, etc. The IO interface (210) serves as the medium where data from the internal logic (e.g., from the microprocessor (208)) transfers to external sources (e.g., the inflation and deflation units, the display (134), etc.). The IO interface (210) mainly serves as a form of input-output communication outside user interaction. Essentially, the IO interface (210) serves as the communication link between the processor (130) elements, between the cuff pressure control system (128) components (130, 132, 134), or between the cuff pressure control system (128) and the inflation and deflation units.

The cuff pressure control system (128) shown in this figure is a general representation of elements that may be included within. The above description of included elements is exemplary and the cuff pressure control system (128) is not limited to having only these elements. It is obvious to those ordinarily skilled in the art that such a system may include additional elements in other embodiments such as power supply, audio output, on/off switch, etc. The elements in the cuff pressure control system (128) may also be integrated or discrete depending on the embodiment.

Input (132) in the cuff pressure control system (128) is shown outside the processor (130), although it is obvious to those ordinarily skilled in the art that automatic input (132) exists within the processor (130), which is handled by the IO interface (210). There may be times when the input (132) has to be done manually by the user. The smart pressure control mechanism is electrical in the preferred embodiment, so cuff inflation/deflation is typically done automatically in real-time. However, there may be rare cases when there is an error in the smart pressure control mechanism. For example, the dual-cuff mechanism may continue inflating even after reaching the threshold pressure range, which would stretch and damage the trachea if only automatic input was allowed. Therefore, manual input (132) by the user can act as a kill switch to mitigate overinflation of the dual-cuff mechanism.

In another embodiment, where cuff inflation/deflation is manual, the cuff pressure control system (128) may also just use the display (134) to instruct users on the degree of cuff inflation/deflation. The input (132) can also play a role in the manual operation of cuff inflation/deflation control, as the user may need to input values (e.g., through a keyboard) to inflate the cuffs accordingly.

The processor (130) contains more than one pressure transducer (212). In the preferred embodiment, there are two pressure transducers (212) within the processor (130), each of which is linked to an individual pressure sensor. However, the above description is exemplary and is not limited to only two pressure transducers (212) in other embodiments. In an alternative embodiment, one pressure transducer may be used to collect data from both pressure sensors. In other embodiments, more than one pressure transducer (212) may be included for accommodating a plurality of sensors inside the pilot balloon assembly to measure the cuff pressure.

The preferred embodiment of the cuff pressure control system (128) is designed with the disposability aspect in mind, even though the components make it sufficient as a stand-alone computer. Generally, the components of the cuff pressure control system (128) would be made of mass-manufactured materials (e.g., circuitry, wiring, and power source), where it becomes more cost-effective to dispose of them after a single use. The cuff pressure control system (128) would be prepackaged with the rest of the endotracheal tube apparatus and used when needed. As a result, it is more hygienic since each apparatus is used for one patient only. In another embodiment, the cuff pressure control system (128) can be a more complex machine with more expensive components. Such a machine would be reusable but would be more costly to maintain, and would require sterilization after each use.

FIG. 3 illustrates a horizontal cross-section of the endotracheal tube apparatus inside a trachea in an uninflated state and an inflated state respectively. In particular, a horizontal cross-section of the cuffs (106, 110) and the endotracheal tube (102) is shown in the figure. Sub-figure (a) illustrates a horizontal cross-section of the uninflated state of the endotracheal tube (102) apparatus in a trachea (302). The trachea (302) has an airway (304) between the outer cuff (106) and the lumen of the trachea (302) or tracheal wall. The outer cuff (106) has an interior outer cuff space (306) that radially separates the outer cuff (106) from the inner cuff (110). The inner cuff (110) has an interior inner cuff space (308) that radially separates the inner cuff (110) and the endotracheal tube (102).

Sub-figure (b) illustrates a horizontal cross-section of the inflated state of the endotracheal tube (102) apparatus in a trachea (302). The inner cuff (110) and the inner cuff opening (114) are touching the outer cuff (106). The outer cuff (110) completely touches the lumen to seal the trachea (302). In the inflated state of the endotracheal tube (102) apparatus, the interior inner cuff space (308) of the inner cuff (110) has increased in size.

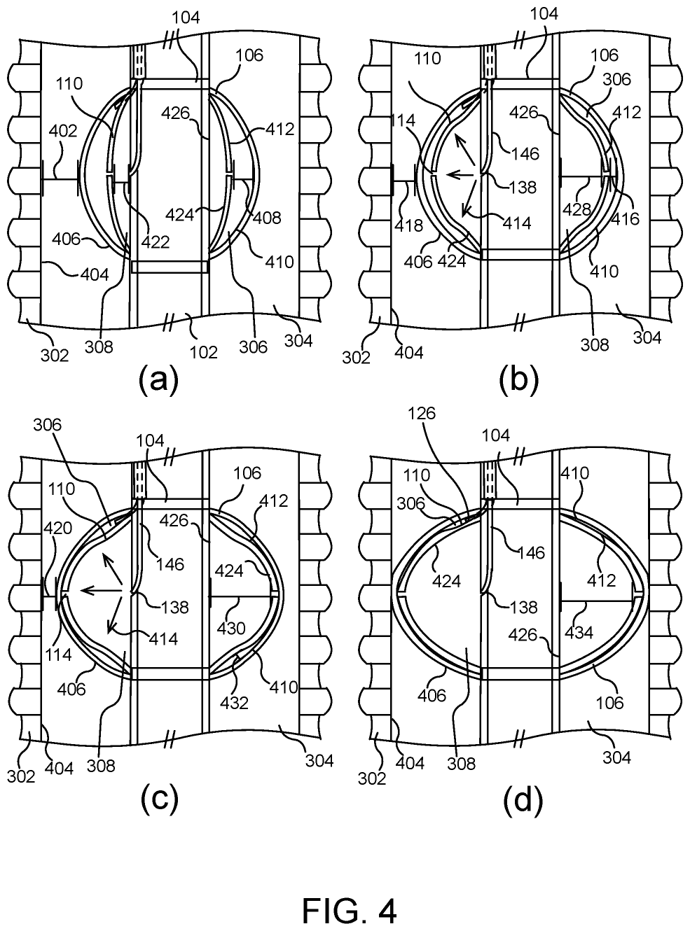

The interior spaces (306, 308) of the cuffs (106, 110) change over time as the cuffs (106, 110) go from the uninflated to the inflated state. It should be noted that the interior spaces (306, 308) are shown at exemplary distances and are not limited to the distances shown in this figure. This will be shown and explained further in FIG. 4.

In addition to the dual-cuff mechanism aspect associated with the cuffs (106, 110), sub-figure (b) illustrates when the cuffs (106, 110) stop inflating right at the lumen of the trachea (302). At the inflated state, the cuffs (106, 110) have reached a threshold in the pressure level (ΔP). This is where the pressure (P1) in the inner cuff (110) is once again greater than the pressure (P2) in the outer cuff (106). In another sense, ΔP has left the pressure equilibrium (P1=P2). As noted earlier, the threshold value is predetermined by a user with prior knowledge of cuff pressure. The smart pressure control mechanism then uses an algorithm to automatically control the components of the apparatus to reach the preset threshold value or a little above that value. Once a pressure level within the acceptable threshold range is reached, the cuff pressure is maintained. If it is away from the acceptable threshold range during the intubation process, then the inflation and deflation units are continuously instructed via the smart pressure control mechanism to carry out a net inflation/deflation function to adjust the size of the cuffs (106, 110) until cuff pressure is at the threshold or within its range. This will be further shown in future figures.

The smart pressure control mechanism helps inflate/deflate the cuffs (106, 110) to the threshold, while the cuffs (106, 110) themselves with the inner cuff opening (114) help determine when the ideal cuff pressure is reached. This is typically done automatically in the preferred embodiment but can be done manually in another embodiment. However, the above description can be considered exemplary and the adjustment of the cuffs to the ideal cuff pressure is not solely limited to such means in other embodiments. In one alternative embodiment, a camera may be installed on the outer cuff (110) for the user to see when that outer cuff (110) touches the trachea (302). Along with the viewing ΔP on the pressure control system, the user can stop cuff inflation/deflation when needed as they view the camera. However, this requires significant device cost, manual effort, and less accuracy in operation, and the user still needs prior knowledge of when to stop cuff inflation/deflation. Furthermore, in the rare event that the apparatus is broken, the camera or any other sensor or probe may be lost inside the patient's trachea (302) and airway (304). In alternative embodiments, other sensors can be used in a similar mechanism and for the same purpose.

FIG. 4 illustrates vertical cross-section views of the dual-cuff mechanism's operation inside the trachea. Sub-figure (a) illustrates the initial state of the dual-cuff mechanism (104) inside a trachea (302). As noted earlier, the interior space of the trachea (302) is the airway (304), which is bounded by the tracheal wall or lumen (404). In this state, there is a starting airway distance (402) between the lumen (404) and the exterior surface (406) of the outer cuff (106). Hereinafter, the ‘exterior surface (406) of the outer cuff (106)’ is referred to as the ‘external outer cuff surface (406)’. The outer cuff (106) has an interior outer cuff space (306) with an initial cuff distance (408) between the interior surface (410) of the outer cuff (106) and the exterior surface (412) of the inner cuff (110). Hereinafter, the ‘interior surface (410) of the outer cuff (106)’ is referred to as ‘internal outer cuff surface (410)’, and the ‘exterior surface (412) of the inner cuff (110)’ is referred to as ‘external inner cuff surface (412)’. The interior inner cuff space (308) has an initial tubal distance (422) between the interior surface (424) of the inner cuff (110) and the exterior side (426) of the tubular body (102). Hereinafter, the ‘interior side (424) of the inner cuff (110)’ is referred to as ‘internal inner cuff surface (424)’, and the ‘exterior side (426) of the tubular body (102)’ is referred to as ‘external tubular surface (426)’.

Sub-figure (b) illustrates the initial expansion of the dual-cuff mechanism (104) inside the trachea (302). Air flow (414) goes through the inner pressure pipe (146), exiting out the inner pressure pipe opening (138) to travel outward in all directions within the interior inner cuff space (308). The interior inner cuff space (308) increases in size during the inner cuff's (110) expansion. A second tubular distance (428) is formed between the internal inner cuff surface (424) and the external tubular surface (426). As the inner cuff (110) expands towards the external inner cuff surface (412), the interior outer cuff space (306) becomes smaller. As a result, a smaller second cuff distance (416) is formed between the external inner cuff surface (412) and the internal outer cuff surface (410). However, the outer cuff (106) also expands, albeit slowly. This is thanks to the inner cuff opening (114)—shown on the left and right sides of the inner cuff's (110) cross-section. This inner cuff opening (114) allows air flow (414) to leak from the interior inner cuff space (308) toward the interior outer cuff space (306). Because the outer cuff (106) is expanding, there is less space within the airway (304). This leads to a second airway distance (418) between the lumen (404) and the external outer cuff surface (406).

Thanks to the inner cuff opening (114), simultaneous inflation/deflation can be achieved. In this sense, only one source of pressurized air, the inner pressure pipe (146), is needed compared to prior arts, where two would normally be required — one for each cuff (106, 110). Although there is an outer pressure pipe that branches away from the inner pressure pipe, the former merely serves as a cover for the inner pressure pipe (146) and as a connecting pathway for measuring pressurized air in the outer cuff (106).

Sub-figure (c) illustrates the dual-cuff mechanism (104) continuing to expand toward pressure equilibrium. Air flow (414) still travels through the inner pressure pipe (146), exiting out the inner pressure pipe opening (138) to travel outward in all directions within the interior inner cuff space (308). The interior inner cuff space (308) continues to increase in size. As a result, a third tubular distance (430) is formed between the internal inner cuff surface (424) and the external tubular surface (426). At this time, the inner cuff opening (114) and most of the external inner cuff surface (412) make contact with the internal outer cuff surface (410). The cuffs (106, 110) are near pressure equilibrium, but the pressure in the inner cuff (110) is still slightly greater than the outer cuff (106). Therefore, small amounts of the interior outer cuff space (306) are still present, shown at the top and bottom sides of the inner cuff (110). A third cuff distance (432) formed between the external inner cuff surface (412) and the internal outer cuff surface (410). The outer cuff (106) continues to expand, shrinking the amount of space in the airway (304) with a third airway distance (420) between the lumen (404) and the external outer cuff surface (406).

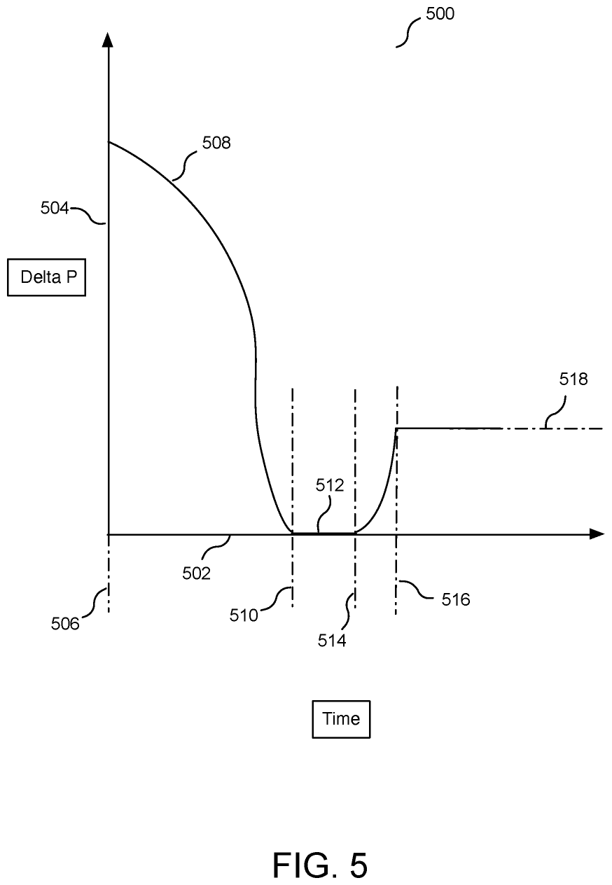

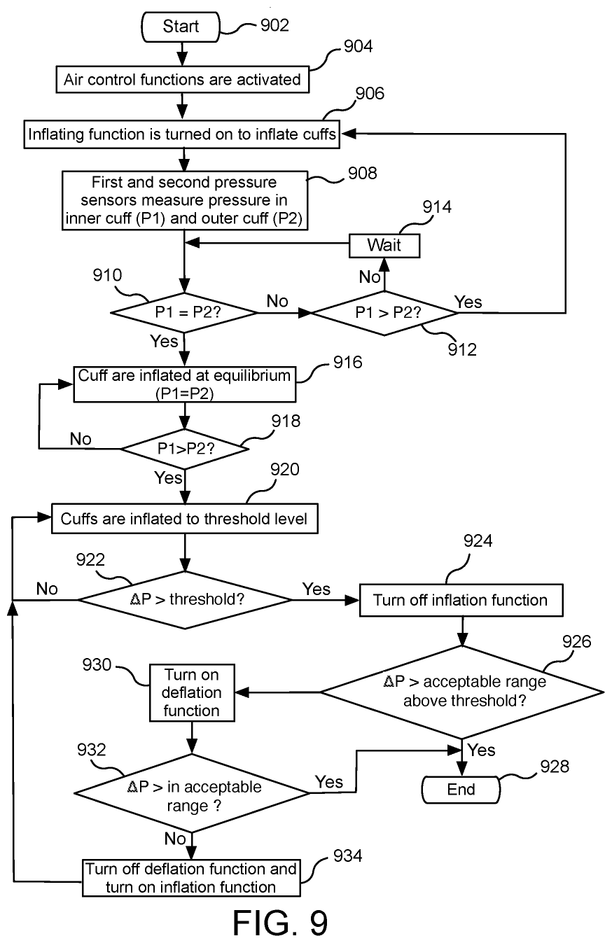

The inner cuff opening (114) allows air flow (414) to inflate the outer cuff (106) to the second (418) and third (420) airway distances. When the inner cuff opening (114) touches the internal outer cuff surface (410), air flow (414) stops entering the interior outer cuff space (306). Rather, the inner cuff (110) now drives the outer cuff's (106) expansion. When this happens, it is an indication that the ideal cuff pressure is close. This will be further shown in future figures, particularly FIGS. 5 and 9.

Sub-figure (d) illustrates a vertical cross-section of the dual-cuff mechanism (104) and associated cuffs (106, 110) in the inflated state. Here, the inner pressure pipe (146) no longer provides pressurized air to the inner cuff (110) via the inner pressure pipe opening (138). The interior inner cuff space (308) is at its largest in the dual-cuff mechanism's (104) inflated state. Here, a final tubal distance (434) is formed between the internal inner cuff surface (424) and the external tubular surface (426). The external inner cuff surface (412) is also touching the distal end of the outer pressure pipe (126). The interior outer cuff space (306) is sealed by the inner cuff (110) except for a small gap around the distal end of the outer pressure pipe (126). The external inner cuff surface (412) is completely touching the internal outer cuff surface (410). The external outer cuff surface (406) now touches the trachea's (302) lumen (404).

The dual-cuff mechanism (104) has an oval shape, with its horizontal center being the part primarily touching the lumen (304) in sub-figure (d). It should be noted that this shape is for exemplary purposes to show the complete sealing of the trachea (302). Therefore, the shape is not limited to that shape. Other alternative embodiments may have the dual-cuff mechanism (104) in a different shape, such as a sphere, a rectangular prism, or any other suitable shape for completely sealing the trachea (302).

It should be noted that the above description of the distances (402, 408, 416, 418, 420, 422, 428, 430, 432, 434) in all sub-figures are for exemplary purposes and serve as a general representation of how far apart the components are from each other. As a result, the distances (402, 408, 416, 418, 420, 422, 428, 430, 432, 434) are not limited to exact measurements. It should also be noted that distances (402, 408, 416, 418, 420, 422, 428, 430, 432, 434) in the sub-figures are placed close to the horizontal center to the dual-cuff mechanism (104) for reference purposes. However, the above description is exemplary and is not limited to the noted locations to demonstrate the distances (402, 408, 416, 418, 420, 422, 428, 430, 432, 434). As the inner (110) and outer (106) cuffs are oval-shaped in the preferred embodiment, the cuff distances (408, 416, 432) may differ at various points between the cuffs (106, 110). For example, in sub-figure (c), there is still a third cuff distance (432) even though much of the inner cuff (110) near the inner cuff opening (114) is touching the internal outer cuff surface (410).

As a cross-sectional figure, the sub-figures show the dual-cuff mechanism (104) expanding on the left and right sides of the dual-cuff mechanism (104) for exemplary purposes to demonstrate cuff inflation. The trachea (302) and lumen (404) are cylindrical. The distances (402, 408, 416, 418, 420, 422, 428, 430, 432, 434) shown in the sub-figures are marked for general visual representation, but because the dual-cuff mechanism (104) expands radially in all directions, these distances (402, 408, 416, 418, 420, 422, 428, 430, 432, 434) can apply anywhere between their respective boundaries. So, the airway distances (402, 418, 420) can be measured from any different area between the lumen (304) and external outer cuff surface (406). The cuff distances (408, 416, 432) can be measured from any different area between the internal outer cuff surface (410) and external inner cuff surface (412). The tubal distances (42, 428, 430, 434) can be any distance between the internal inner cuff surface (424) and the external tubal surface (426).

Looking at sub-figures (b) and (c), air flow (414) is shown exiting the inner pressure pipe (146) on the left side of the apparatus with the inner pressure pipe opening (138) on the left side of the interior inner cuff space (308). However, this is just for the exemplary purpose of showing air flow in the dual-cuff mechanism (104) and particularly, in the interior inner cuff space (308). The air flow (414) that exits the inner pressure pipe opening (138) travels in all directions throughout the interior inner cuff space (308), causing radial expansion of the dual-cuff mechanism (104). Therefore, the placement of the inner pressure pipe (146) and the inner pressure pipe opening (138) is not limited to that particular area within the endotracheal tube (102) apparatus and can be placed anywhere along the cylindrical edge of the endotracheal tube (102) in other alternative embodiments.

The outer pressure pipe (126) is shown exiting the left side of the interior outer cuff space (306). However, this is just for exemplary purposes. Therefore, the placement of the outer pressure pipe (126) is not limited to that particular area within the endotracheal tube (102) apparatus and can be placed anywhere along the cylindrical edge of the endotracheal tube (102) in other alternative embodiments.

It should also be mentioned that although there are two inner cuff openings (114) shown in the figure on opposite sides of the inner cuff (110) (the left and right sides). As noted, this is exemplary and not limited to just those two openings (114) on the inner cuff (110). In other embodiments, the inner cuff opening (114) may take the form of multiple openings or one continuous opening along the horizontal center of the inner cuff (110) depending on the embodiment. This would further exemplify air flow (414) distributing equally throughout the interior inner cuff space (308), allowing for equal inflation of the entire dual-cuff mechanism (104). It should also be noted that although the shape of the inner cuff opening (114) is typically circular, this shape is exemplary. Therefore, the shape of the inner cuff opening (114) is not limited to the circular shape. In other embodiments, the inner cuff opening (114) may be oval, squarish, triangular, or any other shape.

Sub-figure (d) is a visual representation of the dual-cuff mechanism (104) reaching both the threshold level and, eventually, the ideal cuff pressure. As noted earlier, the inner cuff opening (114) can serve as an indicator of when that pressure equilibrium can be reached. The sub-figure is also a visual representation of a threshold level, where the dual-cuff mechanism (104) automatically stops inflating and the cuff pressure is maintained. This will be further shown and explained in FIGS. 5 and 9.

In sub-figure (d), a little bit of the interior outer cuff space (306) is present due to the outer pressure pipe (126) sticking out in the outer cuff (106). In another embodiment, the distal end of the outer pressure pipe (126) can be located right at the external tubular surface (426) so that the outer pressure pipe (126) takes up less space in the interior outer cuff space (306). This would allow for the internal outer cuff surface (410) to completely touch the external inner cuff surface (412) around the illustrated gap area.

In yet another alternative embodiment, the external outer cuff surface (406) may have features or processes that help stabilize the dual-cuff (104.

FIG. 5 illustrates a plot of typical pressure levels within the cuffs over time during cuff inflation. Particularly, the plot (500) measures the cuff pressure difference (508) between the cuffs, also known as ΔP=P1−P2, where P1 is the pressure inside the inner cuff and P2 is the pressure inside the outer cuff, over a period of time. The time is plotted on the x-axis (502), while the difference in cuff pressure level or ΔP (508) is plotted on the y-axis (504). Inflation of the cuffs comprises multiple events at various times that indicate a stage in cuff inflation. At the first event (506) at TO, ΔP (508) is high since the pressure in the inner cuff is much higher than the outer cuff. As inflation begins, however, ΔP (508) begins to drop drastically, up until the second event (510) at T1. At the second event (510), ΔP (508) is zero. This indicates pressure equilibrium (512), where the pressure in the inner cuff is equal to the outer cuff. In other words, the inner cuff completely seals the interior space of the outer cuff. ΔP (508) is kept at equilibrium (512) for a period of time until the third event (514) at T2. At the third event (514), ΔP (508) rises once more. This indicates that the pressure in the inner cuff is larger than the outer cuff. In another sense, the inner cuff is pushing the outer cuff's expansion at the third event (514). ΔP (508) stops rising at the start of a fourth event (516) at T3. Here, ΔP (508) has reached a threshold (518), indicating that the cuffs have expanded to an ideal cuff pressure that optimally seals the trachea. ΔP (508) is kept at that level throughout the fourth event (516) until the end of intubation.

It should be noted that although the plot (500) may show ideal cuff pressure reached always at the threshold (518), the ideal cuff pressure absolute values may be different depending on the patient. A threshold (518) is a constant value that is preset by the user, whereas the actual ideal cuff pressure may depend on the patient themselves and any changing situations that may be present during intubation. This is why the dual-cuff mechanism is designed in a way to achieve an ideal cuff pressure using ΔP (508) measurements from the sensors.

The unit of measurement for ΔP (508) on the y-axis (504) is typically measured in ‘cm H₂O’ or ‘mm Hg’; however, this is only considered exemplary and ΔP (508) is not limited to just that unit of measurement. In other alternative embodiments, ΔP (508) can also be measured in cubic centimeters (cc), or any appropriate unit for pressure depending on the embodiment. The unit of measurement for time on the x-axis (502) is typically in seconds; however, is considered exemplary and time is not limited to that specific unit of measurement. In other alternative embodiments, time may be measured in milliseconds, minutes, or any appropriate form of time depending on the embodiment.

ΔP (508) between the second (510) and third (514) events should be zero, since that is when the cuff pressure difference (508) is zero (where P1=P2), indicating pressure equilibrium (512). However, the ΔP (508) level at pressure equilibrium (512) can also be some value close to zero.

The change rate of ΔP (508) is shown to be non-linear like exponential in this figure. However, this rate of change in ΔP (508) is exemplary and is not limited in that regard. In other embodiments, the cuffs may inflate or deflate at a constant rate, which would be shown as straight diagonal lines on the plot (500).

There may be cases where ΔP (508) may go past the threshold (518) at the start of the fourth event (516). ΔP (508) may be kept at a level slightly above the threshold (518) depending on the inflating unit's control accuracy, which is deemed acceptable for sealing the cuffs. If it is significantly above the threshold (518), the smart pressure control mechanism automatically activates the deflation of the cuffs (via instructions to the deflating unit), which decreases ΔP (508) until it goes back to the threshold (518) level. This negative feedback control has been described previously and will be further shown in FIG. 9.

Although the figure shows ΔP (508) to be constantly maintained at the threshold (518) after the fourth event (516), it may be possible for ΔP (508) to fluctuate during intubation. As a result, the smart pressure control mechanism will automatically control inflation/deflation (via the inflation and deflation units) in order to keep ΔP (508) at the threshold (518) level.

FIG. 6 illustrates a block diagram representation of a general fully automatic closed-loop smart-controlled dual-cuff endotracheal tube. All descriptions of the cuff pressure control system (128) from FIG. 2 815 also apply here. An endotracheal tube (102) and dual-cuff mechanism (104) are inserted into a patient (616) via intubation. The pressure sensors (614), located inside pilot balloons, collect data relating to cuff pressure inside the dual-cuff mechanism (104), which then gets sent to the cuff pressure control system (128). More specifically, the pressure sensors (614) connect and communicate with the pressure transducers (212) inside the processor (130) of the cuff pressure control system (128). The data is processed and analyzed inside the processor (130), specifically through the microprocessor (208). Instructions (204) are then sent out to other elements of the processor like the memory (202), user interface (206), and IO interface (210).

The cuff pressure control system (128) then sends control signals to the inflation (122) and deflation (124) units via a control signal communication (618). Both the inflation (122) and deflation (124) units carry out their respective air control functions (120) to inflate or deflate the dual-cuff mechanism (104) accordingly. The inflation unit (122) has an air supply (602) that stores a volume of air for cuff inflation. The inflation unit (122) has an inflation signal receiver (606), which receives the control signal communication (618) to turn a connected inflation power switch (604) on or off depending on the cuff pressure inside the dual-cuff mechanism (104). The deflation unit (124) has a release valve (608) to expel pressurized air as it travels from the inflation unit (122) to the dual-cuff mechanism (104). The deflation unit (124) has a signal receiver (612), which receives the control signal communication (618) to turn a connected deflation power switch (610) on or off depending on the cuff pressure inside the dual-cuff mechanism (104).

The figure shows the negative feedback loop that governs the smart pressure control mechanism. The communication between the components of the apparatus is typically done automatically in real-time without much manual input (132) from the user; however, this can be manual in another embodiment. The control signal communication (618) in the automatic embodiment is constantly operating during intubation to adjust the inflation/deflation of the dual-cuff mechanism (104) in real-time. This way, a consistent cuff pressure can be maintained in real-time throughout the whole intubation process. Since the feedback loop is electrical in the preferred embodiment, the actions are carried out automatically, making the use of the endotracheal tube (102) apparatus easier since less monitoring is required.

The inflation (122) and deflation (124) units can have discrete power steps that dictate the volume and speed of pressurized air flowing to the dual-cuff mechanism (104). Independent adjustments of the inflation (122) and deflation (124) units allow for numerous combinations of pressurized air flow. This results in increased control accuracy and resolution for the inflation and deflation of the dual-cuff mechanism (104). Because of that, cuff inflation/deflation can be finely tuned to inflate/deflate the dual-cuff mechanism (104) at the required inflating or deflating control level. Whereas an integrated inflation/deflation unit would have a limited number of power step combinations for controlling pressurized air flow to the dual-cuff mechanism (104). For example, an integrated inflation/deflation unit cannot have inflation and deflation turned on at the same time. Whereas, separate inflation (122) and deflation (124) units can be controlled separately so that both inflation and deflation can happen at the same time.

It should be noted that although the inflation (122) and deflation (124) units have discrete power steps in the preferred embodiment, this is for exemplary purpose and not intended to limit to just discrete power steps. In another embodiment, the inflation (122) and deflation (124) units are already continuous in values. In yet another embodiment, the inflation/deflation control is controlled manually without any power source. Several modifications would be present. For example, manual inflation (122) and deflation (124) devices would not include power switches (604, 610) and signal receivers (606, 612), as there are no control signals to send. The instructions (204) in the cuff pressure control system (128) would be modified to merely communicate with other components of the cuff pressure control system (128) like the display (134). No control signal communication (618) would be present in this case.

The pressure sensors (614) are shown to be directly connected to the dual-cuff mechanism (104) as a way to exemplify the connection in obtaining pressure measurement data. As stated in earlier figures, the pressure sensors are located in the pilot balloon assembly in the preferred embodiment. In an alternative embodiment, the pressure sensors (614) can be attached directly inside the dual-cuff mechanism (104) though it might not be preferable.

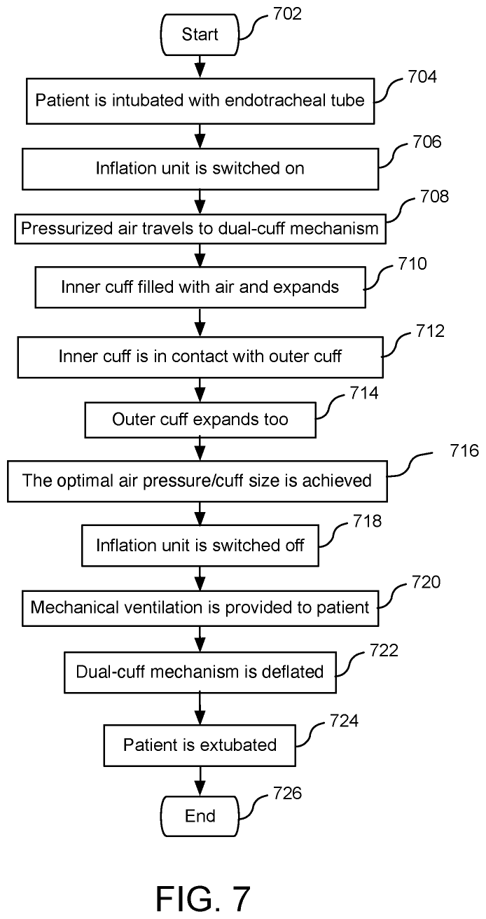

FIG. 7 illustrates a flowchart outlining the intubation process of a patient using the preferred embodiment of the present disclosure. The process starts (702). A patient is intubated with the endotracheal tube at step (704). The inflation unit is then switched on at step (706), which allows pressurized air to travel to the dual-cuff mechanism at step (708). The inner cuff is first filled with air and expands at step (710). Eventually, the inner cuff then comes in contact with the outer cuff at step (712). At step (714), the outer cuff expands too. The optimal air pressure or cuff size is then achieved at step (716). At step (718), the inflation unit is switched off. Mechanical ventilation is provided to the patient for a period of time at step (720). Once intubation is no longer required, the dual-cuff mechanism is deflated at step (722). The patient is extubated at step (724), and the process ends (726).

It should be noted that although the inner cuff is the one that expands at step (710), the outer cuff is also expanding at the same time, albeit at a slower rate. This is thanks to both the inner cuff opening and simultaneous inflation of both cuffs as a result of the dual-cuff mechanism's design.

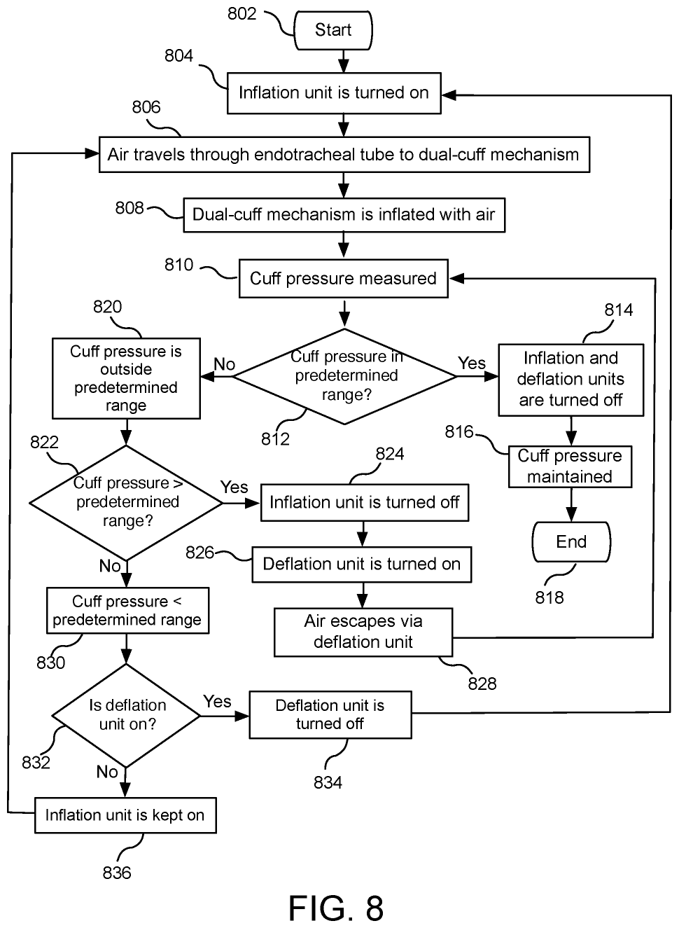

FIG. 8 illustrates a flowchart outlining cuff inflation control logic with the inflation and deflation units. The process starts (802). The inflation unit is turned on at step (804). Pressurized air travels through the endotracheal tube to the dual-cuff mechanism at step (806). The dual-cuff mechanism is then inflated with the pressurized air at step (808). At step (810), the cuff pressure is measured. The cuff pressure is evaluated at step (812) to determine if it is at the predetermined range. If it is not, then it is determined that cuff pressure is outside the predetermined range at step (820). At step (822), the process determines if the cuff pressure is greater than the predetermined range. If it is, then the inflation unit is turned off at step (824). The deflation unit is turned on at step (826), where air escapes via the deflation unit at step (828). The process then moves back to step (810).

If the cuff pressure is not greater than the predetermined range at step (822), it is determined to be lower than the predetermined range at step (830). At step (832), the process determines if the deflation unit is on. If it is, then the deflation unit is turned off at step (834), and the process moves back to step (804). If the deflation unit is not on, then the inflation unit is kept on at step (836), and the process moves back to step (806).

Going back to step (812), if the cuff pressure is within the predetermined range, then the inflation and deflation units (if previously turned on) are turned off at step (814). The cuff pressure is then maintained at step (816), and the process ends (818).

It should be noted that cuff pressure in this figure means the pressure difference (ΔP) between the two cuffs of the dual-cuff mechanism. It should also be noted that the predetermined range refers to the threshold pressure level that triggers the dual-cuff mechanism to stop inflating. This is preset by the user (i.e., medical professional) before the use of the endotracheal tube apparatus.

The figure is a general representation of the method to control pressurized air flow to the cuff using the aspect relating to increased control accuracy and resolution from independent inflation and deflation units. Depending on the type of device, the inflation and deflation units may be turned on and set at discrete power steps to open or close to varying degrees depending on the control signal from the cuff pressure control system; these can be discrete values or a continuous range. The control can also be done manually. In either case, the separation of the inflation and deflation functions means there is greater control of the power step combinations, which allows for precise and accurate air flow to inflate or deflate the dual-cuff mechanism.

In a sense, the figure broadly also exemplifies the smart pressure control mechanism, as air control functions executed by the inflation and deflation units are done automatically based on pressure measurement readings when the cuff pressure is measured at step (810). This will be further shown in the next figure.

FIG. 9 illustrates a flowchart outlining the dual-cuff pressure management mechanism for reaching an ideal cuff pressure. The process starts (902). At step (904), the air control functions are activated. The inflation function is turned on to inflate both cuffs of the dual-cuff mechanism at step (906). During the expansion of both cuffs, the first and second pressure sensors, located inside the air balloon assembly, measure air pressure inside the two cuffs at step (908): the pressure in the inner cuff is measured as P1, while the pressure in the outer cuff is measured as P2. At step (910), the inner cuff pressure (P1) is compared with the outer cuff pressure (P2) to determine if they are equal. If they are not equal, P1 is compared with the P2 to determine if P1 is greater than P2 at step (912). If P1 is greater than P2, then the process moves back to step (906). If P1 is not greater than P2, the user waits at step (914). The process then moves back to step (910).

If P1 is equal to P2 at step (910), then the two cuffs continue to inflate during pressure equilibrium at step (916). At step (918), P1 is compared to P2 to evaluate if P1 is greater than P2. If it is not, the process moves back to step (916). If P1 is greater than P2, the cuffs continue to inflate to a threshold level at step (920). The difference in pressure (ΔP) is evaluated to determine if it is above the threshold level at step (922). If it is not, then the process goes back to step (920). If it is, then the inflation function is turned off at step (924). At step (926), the process evaluates if ΔP is within the acceptable range above the threshold level. If it is, then the process ends (928). If it is not, then the deflation function is turned on at step (930). After some time, ΔP is evaluated to see if it has deflated to an acceptable range at step (932). If it is not, then it has deflated too much; the deflation function is turned off and the inflation function is turned on at step (934). The process then moves back to step (920). If ΔP is within the acceptable range at step (932), the process ends (928).

The figure relates directly to the smart pressure control mechanism of the present disclosure, showing the negative feedback loop as a result of pressure sensor measurements (P1, P2). The inflation and deflation actions of the dual-cuff mechanism are done as a result of ΔP, calculated from P1 and P2. The figure also follows the stages of cuff inflation during pressure equilibrium and when meeting the threshold. However, the figure also relates to the aspect relating to increased control accuracy and resolution, as the air control functions at steps (904, 906, 924, 930, 934) are executable functions carried out by the inflation and deflation units.

It should be noted that while the smart pressure control mechanism does not directly determine ideal cuff pressure or dictate the cuffs to reach the ideal pressure level, the smart pressure control mechanism does instruct the inflation/deflation process, allowing the cuffs to function accordingly to reach ideal cuff pressure for sealing a trachea.

As noted earlier, the pressure in the inner cuff (P1) is almost always greater than the pressure in the outer cuff (P2). However, there are rare cases where P2 is greater than P1, as shown in step (912). Usually, it is sufficient enough to wait for P1 to equal P2 at step (914); however, if the user is waiting for a long time, and P2 is still greater than P1, the inner cuff may be damaged due to a cuff leak. Similarly, pressure equilibrium (P1=P2) may not be reached if the outer cuff may be damaged if P2 is not increasing as P1 increases. In such cases, intubation must stop and the endotracheal tube must be removed from the patient.

It should be noted that the processes shown in FIGS. 7, 8, and 9 are only for exemplary purposes and are not limited to the precise actions in each step, the number of steps, or the order of the steps. In other alternative embodiments, some of such steps may be modified depending on the situation at hand.

1. An endotracheal intubation apparatus for intubating a patient, comprising:
an endotracheal tube having a proximal end, a distal end, and a circumference, the distal end insertable into the patient's trachea for intubating;
an expandable cuff attached around the circumference of the endotracheal tube towards the distal end;
a pressure pipe, a pilot balloon, a balloon sensor, and an inflation and deflation unit; wherein the pilot balloon, balloon sensor, inflation and deflation units are disposed towards the distal end of the endotracheal tube, the pressure pipe pneumatically connects the cuff to the deflation unit and inflation unit via the pilot balloon, the balloon sensor measures the pneumatic pressure of the cuff, the deflation and inflation units are adaptable for respectively deflating and inflating the cuff;
a cuff control system disposed towards the distal end and is electrically connected to the balloon sensor, the power source, deflation unit, and inflation unit; wherein the cuff control system controls the deflation unit and inflation unit to achieve a predetermined pressure in the cuff for sealing space between the patient's trachea and the circumference of the endotracheal tube with the inflated cuff based on the cuff air pressure sensed by the balloon sensor according to a negative feedback control loop scheme.

2. The apparatus of claim 1, further comprising a power source disposed towards the distal end for energizing the endotracheal intubation apparatus.

3. The apparatus of claim 1, wherein the pressure pipe travels along the endotracheal tube; wherein the negative feedback control loop scheme for the cuff control system to maintain the cuff pressure within a predetermined range comprises: activating the deflation unit if the pressure in the cuff increases above the predetermined range and activating the inflation unit if the pressure in the cuff falls below the predetermined range.

4. The apparatus of claim 1, wherein the cuff comprises an inner cuff with an inner cuff opening and an outer cuff encapsulating the inner cuff; wherein the pressure pipe comprises an outer pressure pipe and an inner pressure pipe; wherein the pilot balloon comprises an outer pilot balloon and an inner pilot balloon; wherein the balloon sensor comprises an outer balloon sensor and an inner balloon sensor for measuring the pneumatic pressure of the outer cuff and the inner cuff, respectively; wherein the inner pressure pipe pneumatically connects the inner cuff both to the deflation unit and the inflation unit via the inner pilot balloon, and wherein the outer pressure pipe and the inner pressure pipe pneumatically connect the outer cuff and the inner cuff to the outer pilot balloon and the inner pilot balloon, respectively.

5. The apparatus of claim 4, wherein the pressures of the inner and outer cuff are optimal and determined from the following steps: the inner cuff getting higher inflating pressure due to directly receiving pressurized air from the inflation unit via the inner pressure pipe leading to eventual contact with the inner surface of the outer cuff until it getting sealed and the cuff pressure in both cuffs reaching equilibrium (P1=P2 or DP=0); the inner cuff being inflated more and pushing the outer cuff further to seal the trachea; stopping the inflation at the measured pressure level reaching a threshold.

6. The apparatus of claim 1, wherein the cuff control system monitors the balloon sensor at predetermined intervals to maintain the predetermined pressure in the cuff.

7. The apparatus of claim 1, wherein the shape of the cuff is a donut, triangular, square, or oval.

8. The apparatus of claim 1, wherein the cuff control system activates the deflation unit and/or the inflation unit in discreet steps or in a continuous manner to achieve/maintain the predetermined pressure.

9. The apparatus of claim 1, wherein the deflation unit and the inflation unit are operable and mutually independent of each other.

10. The apparatus of claim 1, wherein the cuff control system simultaneously activates the deflation unit and the inflation unit to maintain the predetermined pressure.

11. The apparatus of claim 1, wherein the cuff control system is operable as a fully automatic smart control system.

12. The apparatus of claim 1, wherein the balloon sensor is adaptable to measure air volume, airflow, and/or temperature.

13. The apparatus of claim 4, wherein the inner cuff opening comprises a plurality of openings.

14. The apparatus of claim 4, wherein the inner cuff opening is circular, oval, square, and triangle in shape.

15. A method for intubating a patient with an endotracheal tube, comprising:
providing an endotracheal tube having a proximal end, a distal end, and a circumference, the distal end insertable into the patient's trachea for intubating;
providing an expandable cuff attached around the circumference of the endotracheal tube towards the distal end;
providing a pressure pipe, a pilot balloon, a balloon sensor, and an inflation and deflation unit; wherein the pilot balloon, balloon sensor, inflation and deflation units are disposed towards the distal end of the endotracheal tube, the pressure pipe pneumatically connects the cuff to the deflation unit and inflation unit via the pilot balloon, the balloon sensor measures the pneumatic pressure of the cuff, the deflation and inflation units are adaptable for respectively deflating and inflating the cuff;
providing a cuff control system disposed towards the distal end and is electrically connected to the balloon sensor, the power source, deflation unit, and inflation unit; wherein the cuff control system controls the deflation unit and inflation unit to achieve a predetermined pressure in the cuff for sealing space between the patient's trachea and the circumference of the endotracheal tube with the inflated cuff based on the cuff air pressure sensed by the balloon sensor according to a negative feedback control loop scheme.

16. The method of claim 15, further comprising providing a power source disposed towards the distal end for energizing the endotracheal intubation apparatus; wherein the pressure pipe travels along the endotracheal tube; wherein the negative feedback control loop scheme for the cuff control system to maintain the cuff pressure within a predetermined range comprises: activating the deflation unit if the pressure in the cuff increases above the predetermined range and activating the inflation unit if the pressure in the cuff falls below the predetermined range.

17. The method of claim 15, wherein the cuff comprises an inner cuff with an inner cuff opening and an outer cuff encapsulating the inner cuff; wherein the pressure pipe comprises an outer pressure pipe and an inner pressure pipe; wherein the pilot balloon comprises an outer pilot balloon and an inner pilot balloon; wherein the balloon sensor comprises an outer balloon sensor and an inner balloon sensor for measuring the pneumatic pressure of the outer cuff and the inner cuff, respectively; wherein the inner pressure pipe pneumatically connects the inner cuff both to the deflation unit and the inflation unit via the inner pilot balloon, and wherein the outer pressure pipe and the inner pressure pipe pneumatically connect the outer cuff and the inner cuff to the outer pilot balloon and the inner pilot balloon, respectively; wherein the pressures of the inner and outer cuff are optimal and determined from the following steps: the inner cuff getting higher inflating pressure due to directly receiving pressurized air from the inflation unit via the inner pressure pipe leading to eventual contact with the inner surface of the outer cuff until it getting sealed and the cuff pressure in both cuffs reaching equilibrium (P1=P2 or DP=0); the inner cuff being inflated more and pushing the outer cuff further to seal the trachea; stopping the inflation at the measured pressure level reaching a threshold.

18. The method of claim 15, wherein the cuff control system monitors the balloon sensor at predetermined intervals to maintain the predetermined pressure in the cuff; wherein the shape of the cuff is a donut, triangular, square, or oval; wherein the balloon sensor is adaptable to measure air volume, airflow, and/or temperature.

19. The method of claim 15, wherein the cuff control system activates the deflation unit and/or the inflation unit in discreet steps or in a continuous manner to achieve/maintain the predetermined pressure; wherein the deflation unit and the inflation unit are operable and mutually independent of each other; wherein the cuff control system simultaneously activates the deflation unit and the inflation unit to maintain the predetermined pressure; wherein the cuff control system is operable as a fully automatic smart control system.

20. The method of claim 17, wherein the inner cuff opening comprises a plurality of openings; wherein the inner cuff opening is circular, oval, square, and triangle in shape.
